(19) United States
(12) Patent Application Publication
(10) Pub. No.: US 2009/0017099 A1
(43) Pub. Date: Jan. 15, 2009

(54) CHOLESTEROL-RICH COMPOSITE FOOD THAT IS RENDERED SUBSTANTIALLY NON-CHOLESTEROLEMIC USING PHYTOSTEROLS

(76) Inventor: Daniel Perlman, Arlington, MA (US)

Correspondence Address: WESLEY B. AMES, 7031 LOS VIENTOS SERENOS, ESCONDIDO, CA, 92029, US

(21) Appl. No.: 11/778,005

(22) Filed: Jul. 13, 2007

(51) Int. Cl. A61K 31/56; A21D 13/00; A61K 47/00

(52) U.S. Cl. 424/439; 426/549; 514/182

(57) ABSTRACT

A composite food is described that includes a single serving portion of a cholesterol-rich food component (abbreviated CRFC) such as a hamburger or hot dog, and a cholesterol-compensating flour-based baked bun configured and sized to hold the CRFC. The bun contains a sufficient amount of phytosterols to compensate for the cholesterol contained in the CRFC, and the phytosterols are provided in a physical and chemical form that is bioavailable during digestion of the composite food.

### RELATED APPLICATIONS

NOT APPLICABLE.

### FIELD OF THE INVENTION

The present invention relates to foods supplemented with phytosterols for reducing dietary cholesterol uptake.

### BACKGROUND OF THE INVENTION

The following discussion is provided solely to assist the understanding of the reader, and does not constitute an admission that any of the information discussed or references cited constitute prior art to the present invention.

Phytosterols such as alpha and beta-sitosterol, stigmosterol, campesterol, and their corresponding fatty acid esters, as well as phytostanols and their esters are derived directly or indirectly from vegetable sources including edible vegetable oils and tall oils. These compounds have proven effective in reducing plasma LDL and total cholesterol levels in humans when included in processed food products that are either cholesterol-free or that contain only low levels of cholesterol. Perlman et al., U.S. Pat. Nos. 6,638,547 and 7,144,595 describe many different processed foods containing varying amounts of fats that may be supplemented with phytosterols. Low fat and fat-free processed foods including fruit juices and yoghurt have also been supplemented with phytosterols.

Pending final written rules, the U.S. Food and Drug Administration (FDA) is allowing foods containing free (non-esterified) phytosterols (as well as esterified phytosterols) to make a health claim (reduces the risk of coronary heart disease when consumed regularly) providing that the food contains at least 400 mg phytosterols per serving, and that the consumer is instructed to consume at least 800 mg free phytosterols per day (the lowest effective daily intake to achieve the stated health benefit). Beyond the original list of allowed foods (fat-based spreads, salad dressings, health bars and dietary supplements), the FDA will allow the health claim for phytosterols in other foods providing those foods meet the following requirements:

1. the food contain at least 400 mg phytosterols or 650 mg phytosterol esters per serving
2. the phytosterol ingredient is at least 80% by weight active material
3. the food meets the following requirements:

(a) it is low in cholesterol, i.e., contains less than 20 mg per serving, and low in saturated fat (less than 1 g saturates per serving) with less than 15% of the food calories coming from saturated fats, and

(b) it contains less than 13 g total fat per serving (or per 50 g of the food product if the referenced serving size is 30 g or less),

(c) it contains less than 480 mg sodium per serving while providing at least 10% of the RDI per serving for at least one of the following: vitamin A, vitamin C, iron, calcium, protein or fiber,

4. for the health claim on the food product, the general term “phytosterols” or “plant sterols” must be used to describe the fortifying substance,
5. the claim on the food product packaging states that the daily dietary intake that may reduce the risk of CHD is 800 mg (or more) of free phytosterols per day
6. The use of the claim otherwise complies with CFR21 S101.83.

A number of processed food products are now being supplemented with free phytosterols and phytosterol esters, and the FDA has mandated that these food products be “heart-healthy” (see above requirements) if the manufacturer wishes to make the above phytosterol-related health claim. Perlman et al., U.S. Pat. Nos. 6,638,547 and 7,144,595 describe many different processed foods containing varying amounts of fats that can be supplemented with phytosterols, and these patents are incorporated herein by reference in their entirety, including all figures and tables.

Notwithstanding currently manufactured food products that contain phytosterols, Applicant is unaware of any composite food product that is configured and arranged to contain a serving of a cholesterol-rich first food component within an edible phytosterol-supplemented second food component that functions to counteract the cholesterol in the first component. In fact, the FDA guidelines for using, and claiming the health benefits of phytosterols in foods teach away from combining phytosterols with more than 20 mg cholesterol or 1 g saturated fat.

### SUMMARY OF THE INVENTION

The presently invented composite foods and methods for reducing gastrointestinal absorption of cholesterol generally utilize a phytosterol-enriched bun with a cholesterol-rich food component such as a hamburger or hot dog. Thus, such “hamburger in a phytosterol enriched bun” and “hot dog in a phytosterol-enriched bun” composite foods contain one serving of a cholesterol-rich food in a baked flour-based carrier bun containing an amount of phytosterol that is sufficient to significantly reduce absorption of cholesterol during digestion of the composite food.

A first aspect of the invention concerns a method for reducing gastrointestinal absorption of cholesterol from digestion of a cholesterol-rich food, and involves ingesting a composite food that includes a single serving portion of a cholesterol-rich food component (CRFC) in a cholesterol-compensating flour-based baked bun. The bun is configured and sized to hold the CRFC and contains a sufficient amount of phytosterols to significantly reduce the absorption of dietary cholesterol into the blood stream of a person from digestion of the composite food.

In particular embodiments, the phytosterols are selected from the group consisting of free phytosterols, fatty acid-esterified phytosterols, corresponding phytostanols, and combinations thereof.

In certain embodiments, the amount of phytosterols in the bun is sufficient to reduce the absorption of dietary cholesterol from the CRFC by at least 20, 22, 25, 27, 30, 32, 35, 37, 40, 42, 45, 47, or 50% compared to the absorption of the cholesterol in a composite food that is the same except that the bun is not enriched in phytosterols.

In some embodiments, the composite food is a complete hamburger, a complete hot dog, a complete meat-containing sandwich, a complete cheese containing sandwich; the CRFC is or includes a hamburger patty, a hot dog wiener, a single serving portion of meat (e.g., sliced meat), beef, pork, poultry, seafood, cheese, or a combination thereof.

Also in certain embodiments, the single serving portion of CRFC contains from 15 to 200 mg cholesterol, from 20 to 150 mg cholesterol, from 20 to 120 mg cholesterol, from 50 to 150 mg cholesterol, from 50 to 100 mg cholesterol, from 75 to 150 mg cholesterol, or from 75 to 120 mg cholesterol; the bun contains at least 300 mg of free phytosterols or at least 490 mg fatty acid esterified phytosterols or an equivalent weighted combination of free phytosterols and fatty acid-esterified phytosterols, at least 400 mg of free phytosterols or at least 650 mg fatty acid-esterified phytosterols or an equivalent weighted combination of free phytosterols and fatty acid-esterified phytosterols, at least 500 mg of free phytosterols or at least 780 mg fatty acid-esterified phytosterols or an equivalent weighted combination of free phytosterols and fatty acid-esterified phytosterols; at least 600 mg of free phytosterols or at least 975 mg fatty acid-esterified phytosterols or an equivalent weighted combination of free phytosterols and fatty acid-esterified phytosterols; at least 700 mg of free phytosterols or at least 1140 mg fatty acid-esterified phytosterols or an equivalent weighted combination of free phytosterols and fatty acid-esterified phytosterols; at least 800 mg of free phytosterols or at least 1300 mg fatty acid-esterified phytosterols or an equivalent weighted combination of free phytosterols and fatty acid-esterified phytosterols; at least 1000 mg of free phytosterols or at least 1625 mg fatty acid-esterified phytosterols or an equivalent weighted combination of free phytosterols and fatty acid-esterified phytosterols; the amount of phytosterols in the bun is in a range defined by taking any two of the preceding specified values for the amounts of free phytosterols or fatty acid-esterified phytosterols or an equivalent weighted combination thereof as endpoints of the range; the weight ratio of phytosterols in the bun to cholesterol in the CRFC is at least 2:1, 3:1, 3.5:1, 4:1, 4.5:1, 5:1, 6:1, 7:1, 8:1, 9:1, or 10:1, from 2:1 to 7:1, from 3:1 to 10:1, from 3:1 to 5:1, from 3:1 to 7:1, from 4:1 to 6:1, from 5:1 to 10:1, from 7:1 to 10:1.

In particular embodiments, the single serving portion of CRFC is at least 50, 75, 100, 125, 150, 175, 200, 225, or 250 g, or is in a range of 30 to 150 g, 30 to 200 g, 50 to 200 g, 50 to 250 g, 75 to 200 g, or 100 to 250 g.

In advantageous embodiments, the phytosterols are provided in a physical and chemical form that is bioavailable during digestion of the composite food.

In further embodiments, the bun is also enriched with an omega-3 rich oil and/or polyphenolic antioxidants, preferably at biologically effective levels.

A related aspect concerns a method for reducing gastrointestinal absorption of cholesterol from digestion of a cholesterol-rich food by ingesting a composite food that includes a single serving portion of a cholesterol-rich food component (CRFC) in a cholesterol-compensating flour-based baked bun. The bun contains at least 400 mg of free phytosterols, or at least 650 mg of fatty acid-esterified phytosterols, or an equivalent weighted combination thereof, and the bun is configured and sized to hold the CRFC.

Embodiments of this method are as described for the preceding aspect (e.g., for the type of composite food or the CRFC, the amounts of phytosterols and/or cholesterol, size of CRFC, the form of the phytosterols, and/or the level of reduction of cholesterol absorption).

Another related aspect concerns the composite food, where the composite food includes a single serving portion of a cholesterol-rich food component (CRFC) and a cholesterol-compensating flour-based baked bun configured and sized to hold the CRFC, and also where the bun contains a sufficient amount of phytosterols to significantly reduce the absorption of dietary cholesterol into the blood stream of a person from digestion of the composite food.

Embodiments of this aspect are as described for the first aspect (e.g., for the type of composite food or the CRFC, the amounts of phytosterols and/or cholesterol, the size of CRFC, the form of the phytosterols, and/or the level of reduction of cholesterol absorption).

Yet another related aspect concerns a composite food that includes a single serving portion of a cholesterol-rich food component (CRFC) and a cholesterol-compensating flour-based baked bun configured and sized to hold the CRFC, where the bun contains at least 400 mg of free phytosterols, or at least 650 mg of fatty acid-esterified phytosterols, or an equivalent weighted combination thereof.

Embodiments of this aspect are as described for the first aspect (e.g., for the type of composite food or the CRFC, the amounts of phytosterols and/or cholesterol, the size of CRFC, the form of the phytosterols, and/or the level of reduction of cholesterol absorption).

In yet another aspect, the invention concerns a method for providing or making a composition food as specified above, by combining a single serving portion of a CRFC with a phytosterol enriched bun in accordance with the description above.

Further, the invention concerns a method for reducing serum cholesterol in an individual by reducing absorption of dietary cholesterol in accordance with the methods described above.

Additional embodiments will be apparent from the Detailed Description and from the claims.

### DETAILED DESCRIPTION OF THE PREFERRED EMBODIMENTS

The present invention concerns a composite food product in which dietary cholesterol that is present as an endogenous constituent in one component of the composite food is compensated and rendered non-cholesterolemic by phytosterols added to a second component of the composite food. In particular, the method involves combining a bakery bun or roll (collectively termed a “bun”) that is sized to contain a hamburger or hot dog, in which the bun contains a substantial amount of phytosterols, e.g., the equivalent of at least 400 mg of free phytosterols or 650 mg of fatty acid-esterified phytosterols. The phytosterols offset or compensate for cholesterol which is present in the meat and/or other components in the composite food, e.g., often between approximately 20 and 120 mg of cholesterol present in a single serving portion of meat in the composite food.

The combined food product (that is, the composite food) including the bun and the meat (and/or other cholesterol-rich) components are eaten as a single food item, and the co-digested components contribute both phytosterol and cholesterol to the gastrointestinal digestive mixture. The amount of phytosterols in the mixture is sufficient to at least greatly reduce absorption of the meat's cholesterol into the blood stream.

It is generally appreciated that dietary cholesterol is contributed by animal fat and other animal tissues such as lean meat, fish, poultry, and milkfat-containing dairy products such as butter, cheese and whole milk-containing foods. For maximum efficacy in reducing cholesterol uptake, phytosterols should be eaten at mealtimes. During digestion, phytosterols (that are negligibly absorbed into the bloodstream) appear to dilute dietary cholesterol, producing so-called “mixed micelles” that are believed to assist in purging cholesterol via fecal elimination, and reducing absorption of the dietary cholesterol.

Such reduction of cholesterol absorption is particular advantageous in view of the high dietary levels of cholesterol commonly ingested in the diets of most individuals in this country as well as others. It has been widely reported that the average American adult male consumes about 300-350 mg cholesterol in his food daily while the average female consumes about 200-250 mg daily. The FDA recommends consuming at least 800 mg of free phytosterols per day, and nutritional researchers suggest as much as twice that level (i.e., about 1.5 g phytosterols per day). Based upon these relative numbers, and knowing that phytosterols can form mixed micelles with cholesterol in the gastrointestinal tract to help in its elimination, Applicant proposes that an amount of free phytosterols be added to a cholesterol-rich food, e.g., where the weight ratio of free sterols to cholesterol in the food is at least 3:1 and preferably 5:1 or 10:1 or greater. If fatty acid-esterified sterols are used in place of free sterols, then their weight ratio (versus cholesterol content of the food) should be greater by approximately 1.6-fold (0.65 g/0.40 g) or a weight ratio of at least 5:1 and preferably 8:1. Of course, a combination of free sterols and fatty acid-esterified sterols can also be used, preferably in a weighted combination according to the just-described weight relationship. In addition, while free phytosterols and fatty acid-esterified phytosterols are the commonly available forms, if other forms of phytosterols which are suitable for dietary use become available, they may also be included in the present methods and compositions. Amounts used may be weighted in the same manner as indicated for free phytosterols and fatty acid-esterified phytosterols based on the respective molecular weights.

### Alternative Methods for Dietary Intake of Phytosterols and Cholesterol-Containing Foods.

1. Admixing Phytosterols with Meats, Cheeses and the Like.

Applicant believes that the public health would be well served if manufacturers of processed foods not only produced more varieties of foods fortified with phytosterols, but also would admix phytosterols into cholesterol-rich foods such as cheeses and processed meat products so that the phytosterols would have maximum efficacy in helping with cholesterol excretion. This efficacy is particularly important because phytosterols are costly (approximately $20.00 per kg or approximately 1-2 cents per serving of food.

With regard to the American and European diet, a significant proportion of dietary cholesterol is consumed in meat products. While it may be difficult to counteract cholesterol by adding phytosterols to intact meat products (e.g., steaks, whole fish and intact poultry products), it would be relatively easy to admix premeasured amounts of phytosterols in appropriate proportions (e.g., 0.4 g-0.8 g phytosterols per 100 g serving of meat or fish) with processed or ground meat products, such as ground beef, pork and poultry that are used in hamburgers, hot dogs, sausages, meat loafs, salamis, and the like.

Notwithstanding this convenient means for introducing dietary phytosterols, Applicant has determined that the U.S. Department of Agriculture (USDA) in concert with the U.S. FDA has established very strict rules limiting what food additives may be added to meat products and what can be stated on the packaging regarding the function of these food additives. For example, while a preservative, antioxidant or flavoring may be added to a meat product and described as such on the packaging, a lengthy petitioning process would be required for phytosterols to be approved for addition to meat, and cited and advertised on the product packaging for the phytosterol's proven ability to reduce plasma cholesterol levels. While technically rational, the governmental regulatory obstacles create a barrier against pursuing this approach to cholesterol reduction. As an additional obstacle, the FDA limitation on asserting the heart health claim for phytosterols requires that a food carrying phytosterols must contain less than 1.0 g of saturated fat and less than 20 mg cholesterol. A 100 g hamburger containing 90% lean meat also contains approximately 10 g beef fat of which almost 4 g is saturated fat. This level of saturates as well as greater than 20 mg of cholesterol in the meat would preclude the heart health claim.

Thus, while the present invention includes such phytosterol-supplemented meat products, current regulations make the commercial provision of such products difficult. For inclusion of phytosterols in processed meat products, the phytosterols can, for example, be incorporated similarly to that described herein for inclusion in buns, that is, the form and amount of phytosterols can be as described for use in the composite foods based on the equivalent amount of meat present or the equivalent ratio of phytosterols to cholesterol. The processed meat product may be, for example, hot dogs, ground meat (e.g., hamburger patties), sausage, salami, bologna, corned beef, and the like. In addition, phytosterols can be incorporated in solid meat products, such as by injection at multiple points, preferably a large number of points. Preferably such incorporation in solid meat products results in phytosterol to cholesterol ratios as described herein for the composite food items. Such solid meat products can include, for example, steaks, hams, roasts, and the like.

2. Phytosterol-Containing Condiment Toppings for Meats.

There are a number of salad dressings and condiments that are being currently manufactured with supplementary phytosterols. While Applicant has not found either commercial tomato ketchup or a mustard fortified with phytosterols, such a product, if it existed, could be applied to hamburgers and hot dogs in an amount sufficient to counteract the cholesterol content within the meat. Such salad dressings and condiments are also within the present invention. Nonetheless, there are at least three drawbacks to producing such condiments for counteracting cholesterol. First, such condiments are often used in large quantities with cholesterol-free foods. For example, tomato ketchup is just as often applied to French fries and used to flavor sauces as it is applied to hamburgers, and mustard is often mixed into potato salad, used in sauces, and applied to sandwiches without meat. Second, the amount of a condiment such as ketchup or mustard applied to a food is a matter of personal choice, and may vary up to about 5-fold depending upon individual taste. This means that some people may add too little phytosterols to balance the cholesterol content of a hamburger or hot dog, while others may add more than necessary. Third, the quantity of a condiment such as ketchup or mustard that is provided to the consumer in typical bottles and jars ranges from 1-3 pounds. Therefore, with a typical serving size being one tablespoon (15 g), there may be 30-90 servings per bottle. If the cost of 400 mg phytosterols is approximately 1 cent, then the direct cost of phytosterol materials for this range of bottle sizes would be 30-90 cents. This represents a prohibitive cost of ingredients.

3. Phytosterol-Containing Buns and Rolls for a Single Serving of Meat.

The primary approach of this invention for providing phytosterols to compensate for the dietary ingestion of cholesterol is through the use of composite foods in which the phytosterols are incorporated in hamburger or hot dog buns. Hamburgers and hot dogs are commonly configured and sized to provide a single serving of meat (which may be in one or more pieces, e.g., hamburgers containing two meat patties) weighing at least 50 g, more typically approximately 100 g, and sometimes as large as 200 g or even more each. While the cholesterol content may vary somewhat depending upon the proportion of fat in the meat, a survey of nutritional ingredient information provided by different commercial suppliers of hamburgers and hot dogs indicates that typical cholesterol levels range from 30 to 50 mg per 100 g meat. Adding cheese may modestly increase these numbers. If a 5:1 weight ratio of free phytosterols to cholesterol is advisable for counteracting a 50 mg quantity of cholesterol in a hamburger or hot dog, then approximately 250 mg free phytosterols (or 400 mg fatty acid-esterified phytosterols) would be advantageous for this purpose. If the standard FDA-recommended single serving amount of free phytosterols (400 mg) is used, then the calculated ratio of milligrams phytosterols to milligrams cholesterol (as contributed by a 100 g hamburger) is excellent (approximately 400:40 or 10:1). Of course, the amount of phytosterols may be adjusted as desired, e.g., to provide a desired phytosterol to cholesterol ratio.

Phytosterols suitable for use in the buns are commercially available from several companies including, for example, Archer Daniels Midland (Decatur, Ill.), Cargill (Minneapolis, Minn.) and Cognis Nutrition and Health (La Grange, Ill.). The phytosterols are commercially available in forms that may be directly blended into bread doughs. For example, free sterols can be purchased as finely divided powders that can be blended either with other dry ingredients or suspended in shortening or vegetable oil that is being added to the recipe. Esterified sterols, on the other hand, can be purchased as fatty pastes or liquids that may be handled and blended like a fat or shortening component in the recipe.

Thus, Applicant herein describes heart-healthy baked buns and rolls (herein collectively termed “buns”) that are sized to be combined, eaten and co-digested with single serving hamburgers, hot dogs and other meat, poultry, fish, and cheese products commonly, e.g., ranging in size from 50 g to 200 g or even a greater range. These serving or portion sizes of cholesterol-containing foods may commonly vary by approximately ±25%. The buns and rolls for use in this invention commonly contain between 400 mg and 800 mg free phytosterols or between 650 mg and 1300 mg fatty acid ester phytosterols per bun or roll. For additional efficacy in reducing plasma cholesterol levels, these phytosterol and phytosterol ester levels may be increased, e.g., by 1.2, 1.4, 1.6, 1.8, or two-fold.

The phytosterol component in the recipe is preferably selected and admixed into the recipe according to methods that assure the bioavailability of the phytosterols as explained elsewhere. For example, in the “Background of the Invention”, Perlman et al., U.S. Pat. No. 7,144,595, describes a number of different invented methods for introducing phytosterols and their esters into foods.

The bakery buns and rolls contain only low levels of fat and sodium, and are easily fortified with at least 10% of the RDI per serving for at least one of the following: vitamin A, vitamin C, iron, calcium, protein or fiber. With such fortification, the buns described herein qualify for the heart health claim, while at the same time providing a sufficient amount of phytosterols to compensate for the endogenous cholesterol in the meat. Since the bun or roll is consumed at the same time as the meat and co-digested, the phytosterols have ample opportunity to mix with the cholesterol and assist in its fecal elimination.

A typical bun as utilized in the present invention commonly weighs 30-60 g or 1-2 oz, has a calorie content of approximately 75-150 calories, and contains between 1.5 and 3 grams of fat. It is conveniently enriched with 10% or more of the RDI of one or more of the above-listed micro-nutrients such as calcium, iron and/or fiber. More specifically, one recipe for a 43 g bun contains 2 g fat from corn oil, 18 g carbohydrate from whole wheat flour, water and also contains small amounts of barley malt, wheat gluten, yeast, salt, and soy lecithin. Any one of hundreds of recipes for buns and rolls is suitable for use in this invention modified for the addition of the phytosterols.

In addition to the phytosterols and micronutrients listed above, additional ingredients can be advantageously added to the bakery bun recipe to further enhance the nutritional profile and health benefits of the composite food. For example, a small amount of omega-3 enriching oil from vegetable (e.g., flax), algae or fish sources can provide DHA, EPA and/or alpha-linolenic acid, thereby providing fortifying omega-3 fatty acids (e.g., 1 to 2 grams) that are lacking in most meat products. Furthermore, any of several different vegetable sources of natural polyphenolic antioxidants that are also lacking in meat products may also be added. These antioxidants are typically provided by, or extracted from the skin, seeds, pulp and/or leaves of grapes, berries and other fruits, vegetables and teas (Camellia sinensis). An example of such a product is the product Activin® from San Joaquin Valley Concentrates, Fresno, Calif., and has 80-90 percent total phenolic content. In particular embodiments, between approximately 50 mg and 300 mg, and preferably between 100 and 200 mg of polyphenolic antioxidants can be beneficially added to a bakery bun of the present invention. Such levels will typically be biologically effective levels. These antioxidants are particularly useful when co-ingested in composite foods containing meats that have been fried, barbecued, or char-broiled at high temperatures. These cooking conditions promote the formation of destructive free radicals. High cooking temperatures lead to the formation of harmful levels of oxygen free radicals, heterocyclic aromatic amines (HAAs) and polynuclear aromatic hydrocarbons (PAHs), also generating destructive free radicals, that are considered carcinogens. The presence of biologically effective levels of polyphenolic antioxidants in composite foods as described herein helps neutralize such co-ingested carcinogens before they are absorbed into the bloodstream.

### Definitions

For terms that are not defined below, the common definition is assumed as provided in the current edition of Webster's International Dictionary or alternatively, provided in a standard organic chemistry textbook such as Organic Chemistry (5^{th} Edition) by Leroy Wade (Prentice-Hall, Inc). Additional terms relating the phytosterols have been defined by Applicant in U.S. Pat. Nos. 6,638,547 and 7,144,595, that are included herein by reference in their entireties. As used in this description and the accompanying claims, the following additional terms shall have the meanings indicated, unless the context requires otherwise:

The term “composite food” as used herein refers to a physically integrated food composition that is generally recognized as including at least two separately prepared component elements, one of which is a single serving portion of food containing a substantial amount of endogenous cholesterol (typically 20 mg or more) such as a hamburger or hot dog. The second component of the composite food is a phytosterol-supplemented bakery bun or roll in which the phytosterols are sufficient to offset the cholesterol contained in the composite food. That is, the phytosterols are provided in a quantity, and in a physical and chemical form that is bioavailable during digestion of the composite food so as to substantially reduce absorption of cholesterol from that composite food.

The term “cholesterol-rich food component” or “CRFC” as used herein, is a food in which the cholesterol content of a single serving of that food is greater than 20 mg. In particular embodiments, the CRFC contains substantially more cholesterol, e.g., at least 30, 40, 50, 60, 70, 80, 90, 100, 120, 140, 150 mg per single serving, or even more.

The term “bun” as used herein refers collectively to any split bread-type baked roll, whether it is a raised bun (such as a hamburger bun that is generally round in shape) or a raised roll (such as a hot dog roll that is generally elongated in shape), and that is of a size sufficient and convenient for holding a single serving or portion of cholesterol-containing meat, poultry, fish or dairy product. Examples of these foods include the hamburger, hot dog, sausage, bologna slices, salami slices, other processed luncheon meat products such as meat loaf, meat balls, animal liver products, chicken and turkey products, cutlets, steaks, chops, fish fillets and cheeses. These single servings of cholesterol-containing foods generally range in weight between 50 g and 200 g. More typically, “quarter pound servings” weigh between 75 g and 150 g.

The term “hamburger” as used herein refers to a patty of ground beef, also known as a beef burger, or simply burger. Hamburger can also refer to the meat itself. This type of meat can be used in combination with non-meat ingredients and extenders such as the American product known as Hamburger Helper®. Ground beef of any form is often commonly referred to as “hamburger.” A quarter pound of ground beef is a typical patty size, but this size may vary upward or downward sometimes by a factor of two. Hamburger does not refer to ham.

The term “hot dog” as used herein refers to a cooked sausage, cured, sometimes smoked, of even texture and flavor that is softer and moister than many other sausages. It is the sausage most readily eaten as finger food in the U.S. It is usually placed hot in a soft, sliced bun of the same general shape as the sausage, and optionally includes condiments and toppings. The flavor of hot dog sausages varies widely by region and by personal preference. Hot dogs are traditionally made from beef and pork or a combination of both meats, although chicken and turkey hot dogs are also available. Unlike many other sausages (which may be sold cooked or uncooked), hot dogs are typically cooked before being offered commercially. Therefore, unless they have spoiled, hot dogs may be safely eaten without further cooking though they are usually warmed up before serving. Hot dogs are also called frankfurters, or franks for short.

The term “cholesterol-compensating” as it refers to the baked bun or bread-type products described herein, refers to the plant sterol (phytosterol) and/or plant stanol (phytostanol) ingredient or a fatty acid ester derivative thereof that is sufficient in both quantity and bioavailability (upon ingestion and digestion together with a “cholesterol-rich food component”) to substantially reduce the amount of cholesterol that is absorbed into the bloodstream from that cholesterol-rich food.

The term “substantially reduce” means that the amount of dietary cholesterol absorbed into the bloodstream from the cholesterol-rich portion of food is reduced by at least 20% as compared to the amount which would be absorbed under a reference condition. For the present invention, the reference condition is ingestion of the cholesterol-rich portion of food with a bun or roll that does not include phytosterols. The absorbed cholesterol and thus the determination of absorption reduction is measured according to either (1) the single isotope method described in Wang et al., 2004, Lipids, 39(1):87-91, or (2) by measuring the ratio of the summed elevations of LDL cholesterol in the blood over basal fasting level following ingestion of a cholesterol-containing food in the presence and absence of included phytosterols. LDL cholesterol is determined at hour time points following ingestion, and LDL cholesterol elevations at the 3 time points most closely bracketing the peak LDL cholesterol levels are summed and used to calculate the ratio.

Likewise, in the present context, the term “sufficient amount of phytosterols to compensate for the cholesterol contained in said CRFC” means an amount of phytosterols included in the bun sufficient to decrease the absorbed cholesterol by at least 20% when measured according to either (1) the single isotope method described in Wang et al., 2004, Lipids, 39(1):87-91, or (2) by measuring the ratio of the summed elevations of LDL cholesterol in the blood over basal fasting level following ingestion of a cholesterol-containing food in the presence and absence of included phytosterols. LDL cholesterol is determined at hour time points following ingestion, and the LDL cholesterol elevations at the 3 time points most closely bracketing the peak LDL cholesterol levels are summed and used to calculate the ratio.

Similarly, the term “substantially non-cholesterolemic” means that the amount of dietary cholesterol absorbed into the bloodstream from the cholesterol-rich portion of food is reduced by at least 20% as compared to the amount which would be absorbed without phytosterols being added to the baked flour-based bun or roll.

The term “phytosterol(s)” as used herein collectively refers to all edible phytosterols and phytostanols. The latter term refers to saturated, hydrogenated, or chemically reduced forms of phytosterols. Collectively, this group of phytosterol substances includes all natural and synthetic forms, isomeric forms and derivatives thereof such as their fatty acid ester derivatives, also known as “ester-sterols” and “phytosterol esters.” For the purposes of the present invention, phytosterols also include any edible agent(s) such as edible surfactants and detergents, e.g., hydrophobic and hydrophilic surfactants such as lecithin, mono and diglycerides, ionic surfactants and the like that may be added along with phytosterols to enhance their solubility and/or dispersibility and/or bioavailability (biological efficacy) in a food. A partial list of phytosterols includes sitosterol, campesterol, stigmasterol, brassicasterol and their corresponding stanols. Additional detail on these agents is provided in the specification of U.S. Pat. Nos. 6,638,547 and 7,144,595 and in the references cited therein.

By mixing the phytosterols (e.g., free and/or fatty acid-esterified) with other bread ingredients and baking them into the bread, they are rendered “physically and chemically bioavailable.” This term means that the phytosterols provided in the buns are able to beneficially function by mixing at the molecular level with cholesterol that is emerging during digestion from the cholesterol-rich foods. After such cholesterol and phytosterol mixing, rather than being absorbed into the bloodstream, it is believed that so-called mixed micelles containing both cholesterol molecules together with a molecular excess of phytosterol molecules, are transported and entrained through the digestive system without being absorbed into the bloodstream. The majority of phytosterol molecules (typically greater than 95%), together with entrained cholesterol molecules, are excreted in the feces.

### EXAMPLE

The following test kitchen recipe for hamburger buns and hot dog rolls (food processor) can be scaled up to commercial batch size using conventional methods known in the art.

Ingredients for Twelve Buns
5 cups all-purpose flour
4 tsp. active dry yeast
1 Tbsp. salt
2 cups warm water
2 Tbsp. shortening
4.8 grams non-esterified phytosterols or 7.8 grams fatty acid-esterified phytosterols (CardioAid® brand phytosterols obtained from ADM Nutritionals, Decatur, II)

Directions:

Dissolve yeast in 0.5 cups of water with a 0.5 tsp sugar, and let proof for 10 minutes.

Place flour and salt in food processor and pulse twice briefly to blend. Add shortening pre-blended with phytosterols, and pulse several times to cut in.

With the processor running, add dissolved yeast and the remaining 1.5 cups water in a steady stream. When dough forms a ball on blade, let the machine run 45 seconds to knead. Turn dough out of the work bowl onto lightly floured surface and knead until dough is very smooth.

Place dough in an oiled bowl and let rise about an hour, or until double.

Turn risen dough out onto work surface and do either of the following:

For Round Hamburger Buns:

Cut the dough into 12 pieces and manually roll each piece until smooth. Pinch the dough together on the bottom and place the rolls on a greased baking sheet.

For Elongated Hot Dog Rolls:

Cut the dough into 4 chunks. Manually roll each chunk into a cylinder about 1.5 inches thick and about 15 inches long. Cut the cylinder into 3 pieces. Flatten each piece slightly, and form a crease lengthwise down the middle of each piece. Fold the dough along the crease and pinch the edges together. Place on greased baking sheet.

Cover and let rise for 40 minutes; Preheat oven to 400 degrees F.

Before baking, optionally brush or spray rolls with milk and sprinkle with sesame seeds.

Bake about 20-22 minutes, until puffed and golden brown.

Other brands of vegetable oil-derived and tall oil-derived phytosterols and phytosterol esters may be substituted into this recipe and other recipes for buns, rolls and the like, and are within the scope of the present invention and the following claims.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the amounts of phytosterols used, the form of the phytosterols, and the cholesterol-rich food. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

What is claimed is:

1. A method for reducing gastrointestinal absorption of cholesterol from digestion of a cholesterol-rich food, comprising
ingesting a composite food that comprises a single serving portion of a cholesterol-rich food component (CRFC) in a cholesterol-compensating flour-based baked bun,
wherein said bun contains a sufficient amount of phytosterols to significantly reduce the absorption of dietary cholesterol into the blood stream of a person from digestion of said composite food and wherein said bun is configured and sized to hold said CRFC.

2. The method of claim 1, wherein said sufficient amount of phytosterols is sufficient to reduce the absorption of dietary cholesterol by at least 20%.

3. The method of claim 1, wherein said sufficient amount of phytosterols is sufficient to reduce the absorption of dietary cholesterol by at least 30%.

4. The method of claim 1, wherein said composite food is a complete hamburger.

5. The method of claim 1, wherein said composite food is a complete hot dog.

6. The method of claim 1, wherein said CRFC is selected from the group consisting of meat, poultry, seafood, cheese and combinations thereof.

7. The method of claim 1, wherein said single serving portion of CRFC contains between approximately 20 mg and 120 mg of cholesterol.

8. The method of claim 1, wherein the weight ratio of phytosterols in said bun to cholesterol in said CRFC is at least 3:1.

9. The method of claim 1, wherein the weight ratio of phytosterols in said bun to cholesterol in said CRFC is at least 5:1.

10. The method of claim 1, wherein said bun contains at least 400 mg of free phytosterols, or at least 650 mg of fatty acid-esterified phytosterols, or an equivalent weighted combination thereof.

11. The method of claim 1, wherein said bun contains at least 500 mg of free phytosterols or at least 780 mg of fatty acid-esterified phytosterols or an equivalent weighted combination thereof.

12. The method of claim 1, wherein said bun contains at least 800 mg free phytosterols or at least 1300 mg fatty acid-esterified phytosterols or an equivalent weighted combination thereof.

13. The method of claim 1, wherein said phytosterols are provided in a physical and chemical form that is bioavailable during digestion of said composite food.

14. The method of claim 1, wherein said bun is supplemented with an oil rich in omega-3 fatty acids or with polyphenolic antioxidants or both.

15. A method for reducing gastrointestinal absorption of cholesterol from digestion of a cholesterol-rich food, comprising
ingesting a composite food that comprises a single serving portion of a cholesterol-rich food component (CRFC) in a cholesterol-compensating flour-based baked bun, wherein said bun contains at least 400 mg of free phytosterols, or at least 650 mg of fatty acid-esterified phytosterols, or an equivalent weighted combination thereof and said bun contains is configured and sized to hold said CRFC.

16. The method of claim 15, wherein said phytosterols are provided in a physical and chemical form that is bioavailable during digestion of said composite food.

17. The method of claim 13, wherein said bun contains at least 500 mg of free phytosterols or at least 780 mg of fatty acid-esterified phytosterols or an equivalent weighted combination thereof.

18. The method of claim 13, wherein said bun contains at least 800 mg free phytosterols or at least 1300 mg fatty acid-esterified phytosterols or an equivalent weighted combination thereof.

19. A composite food, comprising
a single serving portion of a cholesterol-rich food component (CRFC) and a cholesterol-compensating flour-based baked bun configured and sized to hold said CRFC, wherein said bun contains a sufficient amount of phytosterols to significantly reduce the absorption of dietary cholesterol into the blood stream of a person from digestion of said composite food.

20. A composite food, comprising
a single serving portion of a cholesterol-rich food component (CRFC) and a cholesterol-compensating flour-based baked bun configured and sized to hold said CRFC, wherein said bun contains at least 400 mg of free phytosterols, or at least 650 mg of fatty acid-esterified phytosterols, or an equivalent weighted combination thereof, and wherein said phytosterols are provided in a physical and chemical form that is bioavailable during digestion of said composite food.
